(19) United States
(12) Patent Application Publication
(10) Pub. No.: US 2007/0232986 A1
(43) Pub. Date: Oct. 4, 2007

(54) Low-profile implantable ultrasound array and method for enhancing drug delivery to tissue

(75) Inventors: Axel F. Brisken, Fremont, CA (US); Joseph Karratt, Mountain View, CA (US); Gill Heart, Atlanta, GA (US)

Correspondence Address: Kirkpatrick & Lockhart Preston Gates Ellis LLP;(FORMERLY KIRKPATRICK & LOCKHART NICHOLSON GRAHAM), STATE STREET FINANCIAL CENTER, One Lincoln Street, BOSTON, MA, 02111-2950, US

(73) Assignee: CytoDome, Inc., Atlanta, GA (US)

(21) Appl. No.: 11/413,652

(22) Filed: Apr. 28, 2006

Related U.S. Application Data
(60) Provisional application No. 60/788,424, filed on Mar. 31, 2006.

(51) Int. Cl. A61B 17/20

(52) U.S. Cl. 604/22

(57) ABSTRACT

An implantable ultrasound transducer device is defined by a plurality of coaxially, longitudinally spaced and acoustically isolated transducer elements, controlled as a phased array to define an exposure annulus within tissue disposed about the transducer array. Operation of the array is controlled to enhance transport of therapeutic substance through the tissue while avoiding thermal damage. The array is configured to enable it to be inserted into the tissue and removed from the tissue through a small surgical or natural opening.

### FIELD OF THE INVENTION

This invention relates to implantable ultrasound transducer devices and their use to enhance delivery of therapeutic substance to tissue.

### BACKGROUND

The extent to which therapeutic substances can be delivered, transported to and taken up by internal targeted tissue can be enhanced by the selective application of ultrasound energy from an implanted source. In particular, local application of therapeutic substances within a defined ultrasound field that emanates from a source in immediate proximity to the target tissue enhances transportation and uptake of therapeutic molecules at a rate and over a distance substantially greater than is achieved by natural diffusion. Such improvements are described in U.S. patent application Ser. Nos. 10/746,311 filed Dec. 24, 2003 and 11/140,845 filed May 31, 2005. The disclosures of those applications are herein incorporated by reference as part of the present specification.

The ultrasound energy developed by the transducer mostly a function of the intensity of emission of the transducer and the area of its radiating surface. Therefore, the transducer must be of a sufficient size to generate an ultrasound field with characteristics capable of enhancing the transport of therapeutic molecules through the tissue and be taken up by the cellular elements of the tissue at rates and in a concentration substantially greater than those resulting from natural diffusion. However, it is desirable, generally, to minimize the size of the surgical opening in connection with any surgical operation and, in the context of the field of the present invention, the size of the ultrasound transducer or transducer array for generating a field for enhancing drug delivery might be a large size opening. One example is in connection with treatment of the brain with therapeutic substances, typically requiring surgical access through the skull. Where it is desired to enhance the delivery of the therapeutic substance, it would be desirable to minimize the extent of opening that must be formed through the skull, both for insertion of the device as well as its removal, and without compromising the ability of the ultrasound transducer to develop sufficient energy.

One approach for placing a transducer through a small opening may be to construct the transducer in separate components so that each can be inserted separately through the opening and then reassembled internally of the patient. Such an approach has been proposed, for example, in connection with high energy ultrasound treatment of benign prostate hypertrophy, described in U.S. patent application Ser. No. 10/452,061, filed Jun. 2, 2003, Pub. No. 2004/0242999. It would be preferable to avoid the complexities of construction and use of such a device. Therefore, it is an object of the present invention to provide a device and methods by which a therapeutic drug can be delivered locally to a specified region of tissue, such as brain tissue, and in which a unitary ultrasound transducer can be inserted and removed through a relatively small opening while being able to develop an ultrasound field of sufficient power to enhance both transport and uptake of therapeutic substances.

### SUMMARY OF THE INVENTION

The invention includes the use of a multi-element piezoelectric transducer array of substantially uniform cross-section (e.g., circular) mounted to the distal region of a catheter or other probe element adapted to be inserted and navigated through a natural or surgically formed opening of a patient in a relatively minimally invasive procedure. The multi-element transducer array is configured and is operated to generate a radially propagating ultrasound field about the array. The system is operated as a phased array and is controlled to generate an annular exposure region about the array that includes a focal ring radially spaced from the emitting surface of the array. The transducer elements are driven in phased relationship to cause constructive interference of the emitted ultrasound pulses to define the focal region. The field is defined by a radially outward, progressively increasing, degree of constructive interference of the ultrasound waves from the emitting surfaces of the transducer elements, resulting in the annular focal ring. Consequently, the amplitude of the resultant acoustic pressure wave will increase as the wavefront approaches the focal zone, resulting in progressively increasing acoustic pressure pulses being applied to therapeutic molecules and tissue components within the annular field. Because the amplitude of the resultant pressure wave increases as it propagates radially outwardly from the transducer array, the amplitude of the pulses emitted at the radiating surface of the transducer elements may be maintained at a sufficiently low level to avoid adverse thermal effects on the surrounding tissue while providing a threshold level of acoustic pressure sufficient to enhance the transport and uptake of the substance.

The elements in the transducer array are acoustically isolated from each other so that each of them can be controlled to generate the focal region and its location. The array may be driven by a signal generator controlled by a computer that also controls operation of phase shifters and amplifiers that drive the individual transducer elements. The computer controls the operating frequency of the signal generator and the operation of the phase shifters which enable the acoustic beam to be steered and to adjust the diameter of the exposure annulus. The computer also controls adjustment of the power levels of the amplifiers which drive the transducer elements so as to control the output emission amplitude of the transducer elements.

The elements in the array may be disc-shaped and may be mounted on a tubular core. Wiring to transmit electrical pulses to the individual transducer elements may be passed through the core tube. The core tube also may be employed to direct therapeutic substance, such as drugs, to the regions between the isolated transducer elements from which they may be emitted radially outwardly of the transducer and exposed to the targeted tissue.

The transducer array may be cylindrical and may have a uniform cross-sectional dimension (e.g., circular) along its length so that it may be inserted through a relatively small opening (e.g., in the skull) and also removed through that opening.

### DESCRIPTION OF THE DRAWINGS

Objects and advantages of the invention will be appreciated more fully from the following description, with reference to the accompanying drawings wherein:

FIG. 1 is a diagrammatic illustration of a device having a transducer array at its distal region in accordance with the invention;

FIG. 2 is an enlarged, diagrammatic longitudinal cross-section of the ultrasound array.

FIG. 3 is a sectional illustration of a disc-like ultrasound transducer element;

FIG. 4 is an illustration of a control circuit for operating the phased ultrasound array;

FIG. 5 is a diagrammatic illustration of the ultrasound field generated by the phased array and including a sweepable annular focal region; and

FIG. 6 is a diagrammatic illustration of the core tube and several spacers illustrating the manner in which drugs or other substances can be delivered through passageways to the region of tissue about the device;

FIG. 7 is an illustration of another embodiment of the device in which separate channels are provided for the ultrasound wires and for the substance delivery; and

FIG. 8 is a diagrammatic, sectional illustration as seen along the line 8-8 of FIG. 7.

### DESCRIPTION OF THE PREFERRED EMBODIMENT

FIG. 1 illustrates, diagrammatically, a device in accordance with the invention that includes a shaft-like delivery structure 10, such as a catheter, a probe, or other device adapted to be inserted into a patient, either through a natural or surgically made opening and advanced to an intended treatment site adjacent to or within targeted tissue. An ultrasound transducer array is mounted at the distal end 12 of the shaft 10. The shaft 10 typically may be tubular having at least one central lumen that may contain wires or form a pathway through which a guidewire or therapeutic substances may be deployed. A fitting 14 may be provided at the proximal end of the shaft 10 and may include appropriate electrical connections for the transducer wiring and access to one or more lumens that may extend through the device.

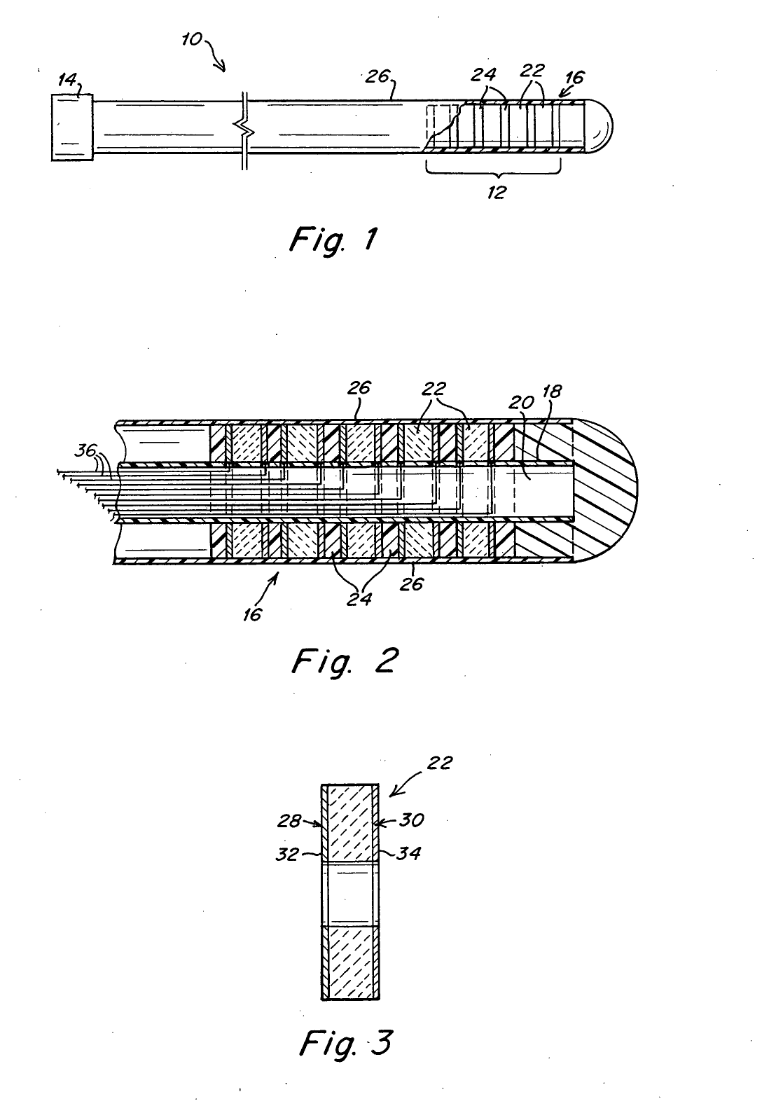

FIG. 2 illustrates, in diagrammatic cross-section, the configuration of an embodiment of a transducer array 16 in accordance with the invention. The transducer array 16 may include a central core tube 18 that defines a central lumen 20 communicating with a lumen in the main shaft 10. The core tube 18 preferably is formed from a polyimide or other suitable material and serves as a support for a plurality of disc-like annular transducer elements 22. The transducer elements 22 have central apertures by which they are mounted about the core tube so that they are acoustically isolated from each other. Each of the transducer elements 22 may be bonded to a small amount of an appropriate adhesive such as a low viscosity epoxy. Preferably, the adhesive should be placed sparingly and should not fill the facing surface of the tube 18 and transducer elements 22. The transducer elements 22 are spaced along the length of the core tube 18, the spaces providing acoustic isolation of the individual elements 22. Alternately, the spaces may be filled with non-acoustically conductive spacers 24. By way of example, spacers formed from expanded polytetrafluoroethylene (e-PTFE), epoxy foam, silicone rubber foam or other material that provides acoustic insulation, may be employed. Further, the spacers between the piezoelectric discs may be cut to one quarter wave length in thickness so as to further diminish the communication of acoustic energy from one element to the next.

In some embodiments, the spaces between the ultrasound elements 22 may be employed as a pathway to enable therapeutic substances to be delivered from the core tube 18 to the outer surface of the array. This might be accomplished by induced porosity in the filler material or by the implantation or formation of discrete micro lumens.

The outer surface of the array may be covered by a polymeric jacket 26 such as, for example, Pebax, low density polyethylene or the like, that may be shrunk onto and about the transducer elements 22 and spacers 24. The jacket may be porous or apertured to enable delivery of liquid substances to the target tissue. The outer cylindrical surfaces of the transducer elements 22 may be bonded to the inner surface of the outer jacket 26 as by an epoxy adhesive layer. The outer jacket may typically be a small fraction of a wavelength in thickness and serves mainly as a bio barrier between the components of the probe assembly and human subject tissues.

As shown in FIG. 3 each of the transducer elements 22 may be considered as having a proximal face 28 and a distal face 30 and each of those faces may be coated or otherwise formed to include a conductive electrode surfaces 32, 34. Wires 36 extending through the core tube 18 are attached to the electrode surface 32, 34 for each transducer element, there being a separate pair of wires associated with each individual transducer element 22 so that each element 22 can be controlled independently to shape and vary the acoustic field about the transducer array.

The transducer elements 22 preferably are formed from a piezoelectric material selected for power delivery applications, such as PZT-8 or PZT-4. The individual elements 22 may, for example, by of the order of about 8 mm in diameter and about 2 mm thick for a radial resonant frequency of the order of about 150 kHz. The spaces between the facing surfaces of adjacent elements 22 may be of the order of about 1 mm. The elements 22 may exhibit a thickness resonant frequency of about 1.0 MHz.

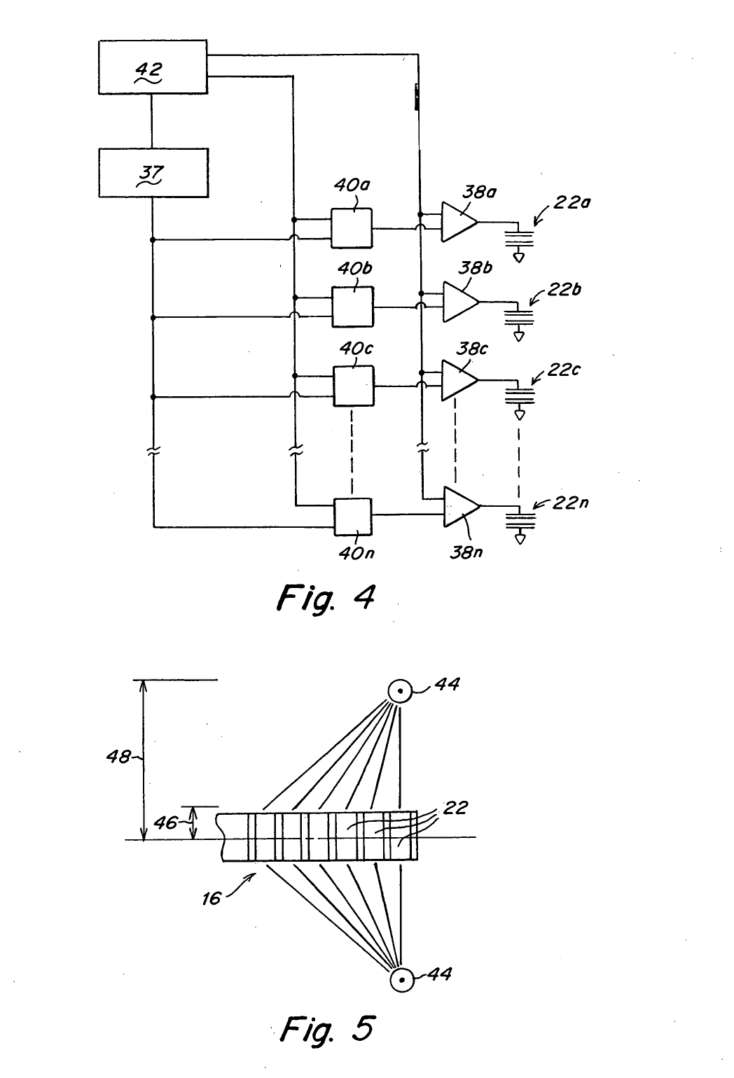

FIG. 4 illustrates, diagrammatically, a control system for operating the transducer array. Each transducer element 22a, 22b . . . 22n is driven by a signal from a signal generator 37. The signal is amplified by a computer-controlled amplifier 38a, 38b . . . 38n and the timing of each signal is controlled by a phase shifter 40a, 40b . . . 40n that also is controlled by the computer 42. The phase shifters are interposed in circuit with the computer-controlled output from the signal generator. The computer 42 selects the operating frequency of the signal generator 37 and controls the phase shifters 40 to steer the acoustic beam and adjust the diameter of the exposure annulus. It is anticipated that acoustic emission for each target area be in the form of bursts of acoustic energy, where each burst may have a duration of typically 5 to 100 cycles. It is further anticipated that the transmit array may be operating continuously, or with the highest possibly duty cycle, directing annular beams to various target regions. The target regions thus receive acoustic emission with a duty cycle approximately equivalent to the transmitting array duty cycle approximately equivalent to the transmitting array duty cycle divided by the number of target annuli.

FIG. 5 illustrates, diagrammatically, the manner in which the cylindrical phased array functions to generate an annular ultrasound field about the transducer array 16. The field includes an annular focal ring 44 at which the ultrasound pulses from the transducer elements 22 constructively interfere to define a region at which the resultant amplitude of the interfering waves is at a maximum. The position of the focal ring 44 is a function by the phase delays of the pulses of the individual transducer elements 22 which are controlled by the computer 42, to enable the focal ring 44 to be swept through an annular range that extends radially about the transducer array from an inner radius 46 to an outer radius 48 as well as longitudinally of the array. The degree of constructive interference of the ultrasound waves and, therefore, the intensity of the ultrasound energy to which surrounding tissue is exposed, will increase progressively in a radially outward direction. Thus, the amplitude of the signals emitted from the transducer elements 22 can be of a lesser amplitude than the resultant amplitude that will develop in the region of the focal ring. Consequently, the risk of exposing tissues adjacent the surface of the transducer elements 22 to higher amplitude signals and possible thermal damage is reduced.

The thickness cross-sectional dimensions of the ring end 44 (thickness and depth of field) depend on the operating frequency, the diameter of the array and the length of the array. By way of example, a 1 cm diameter, 2 cm long array operating at 1 MHz might be programmed to create an annular ring at 2 cm from the surface with a length of 3 mm and a radial thickness ΔR of approximately 3 mm. In this example, with a radiative surface area of 6.2 cm² the exposure surface area is approximately 4.7 cm², enabling the surface amplitude to be less than that at the focal ring.

Alternatively, instead of operating the cylindrical array in a phased array mode wherein the acoustic focal zone annulus is swept back and forth parallel to the cylindrical axis, and possibly radially outward back and forth, the array may be operated with all phases identical, in which case the array appears as a sparsely populated cylindrical radiator. In this case, the highly focused annular focal ring becomes a more diffuse annular ring at a greater distance from the cylinder. Further, the acoustic field will fall off more strongly as the reciprocal of the distance from the surface. This mode might be more beneficial in the movement of drugs closer to the surface of the device, as compared to the focal annular ring which would move drugs at a greater distance. Due to the system architecture, this “unfocused” mode may be programmed into the system with or instead of a sweeping focused mode.

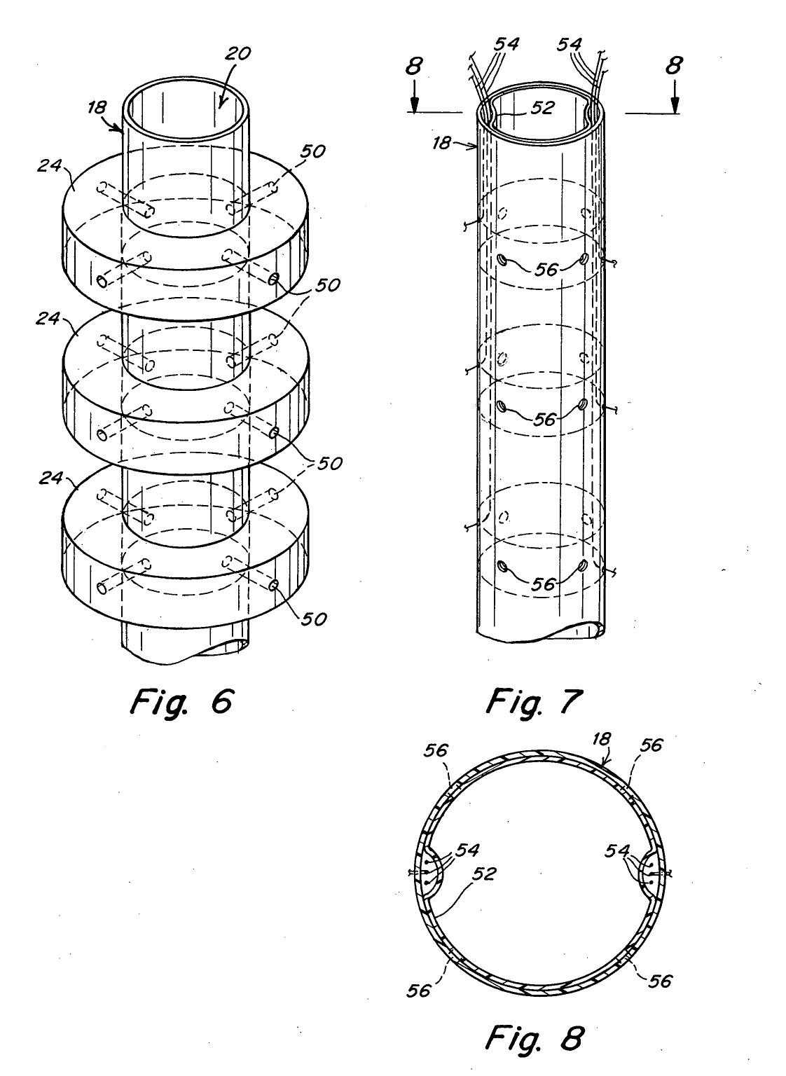

FIG. 6 illustrates, diagrammatically and with transducer elements and conductors omitted for clarity, an arrangement of lumens and passageways by which therapeutic substances may be directed through the device to be delivered to the tissue immediately about the transducer array. The drawing shows the relationship of the core tube 18 with several of the spacers 24 mounted in longitudinal spaced relation along the core tube 18. Radial passageways 50 may be formed, as by laser drilling, through the core tube 18 and the spacers 24 to enable substances to flow through the core tube and then radially outward to the periphery of the device. The outer jacket 26 (omitted for clarity in FIG. 6) may be porous or also may be formed with radial holes through which the substance can be emitted.

FIGS. 7 and 8 illustrate another embodiment in which separate passageways are provided for the transducer wires and for substance delivery. In this embodiment an inner tube 52 is provided within the core tube 18. The transducer wires 54 may be captured between the inner and out tubes and may be gathered in bundles so that the majority of the cross-sectional periphery of the composite core-delivery tube 18, 52 is defined by the adjacent walls of the inner and outer tubes. Apertures 56 may be laser drilled through the regions between the wire bundles to communicate with apertures or other passageways formed in the spacers, ultimately providing a passageway through the delivery channel, the spacers mounted about the core tube, and the outer jacket.

From the foregoing, it will be appreciated that a transducer system in accordance with the principles of the invention is particularly suited for low profile applications in which the unitary device can be inserted and removed through a relatively small natural or surgically formed opening. For example, the device may be employed to treat regions of the brain by making a relatively small opening in the skull through which a device, having a fixed maximum cross-section, can be inserted and removed with relative ease.

It should be understood that the foregoing description of the invention is intended merely to be illustrative thereof and that other embodiments, modifications and equivalents may be apparent to those skilled in the art without departing from the scope or spirit of the invention.

1. An apparatus for enhancing delivery of therapeutic substances to a region of tissue comprising:
a multiple-element array of coaxially aligned, acoustically isolated, annular ultrasound elements, the array defining a fixed maximum outer diameter adapted to enable insertion and removal of the array through a body opening of predetermined size, each ultrasound element having an outer radiating surface for emitting ultrasound energy in a radially outward pattern about the array;
a signal generator adapted to provide signals to activate the ultrasound elements;
phased array control means for controlling the relative timing of signals transmitted to the ultrasound elements;
the signal generator and control means being adapted to cause the transducer array to emit ultrasound pulses to define an annular exposure field that includes an annular focal ring of constructively interfering ultrasound energy spaced radially outward of the emission surface of the array;
the intensity of the ultrasound energy field about the transducer progressively increasing from the transducer surface radially outward toward the focal ring; and
whereby the ultrasound energy from the surface of the transducer array outward to the annular focal ring may be controlled to be sufficient to induce movement of substance molecules through tissue about the array in a radially outward direction but below a level that would cause thermal damage to the tissue.

2. An apparatus as defined in claim 1 wherein the multiple element array has a uniform cross-section along its length.

3. An apparatus as defined in claim 1 wherein the array is cylindrical.

4. An apparatus as defined in claim 1 wherein the ultrasound energy field is characterized by a frequency in the range of about 100 kHz to about 1,500 kHz, a mechanical index of between about 0.5 and about 3.0, the ultrasound being pulsed and having between about 5 to about 100 cycles per pulse and a pulse repetition frequency of between about 100 to about 10,000 Hz.

5. An apparatus as defined in claim 1 wherein the ultrasound energy at the surface of the transducer is such that the tissue is not raised more than about 2° C.

6. An apparatus as defined in claim 1 further comprising:
an acoustically dampening material disposed between adjacent annular ultrasound elements.

7. An apparatus as defined in claim 6 wherein the damping comprises at least one of air, epoxy foam, silicone rubber foam or expanded polytetrafluoroethylene.

8. A device as defined in claim 1 further comprising:
an elongate core tube;
each of the ultrasound elements in the array being mounted to and along the core tube in secured, spaced relation;
a plurality of conductors extending through the core tube, the conductors being arranged in pairs, each pair being electrically connected to one of the ultrasound elements.

9. An apparatus as defined in claim 8 wherein each of the ultrasound elements has a proximal face and a distal face, each of which is surfaced with an electrode, each electrode being connected to one of the conductors in a pair of conductors.

10. An apparatus as defined in claim 1 further comprising a radiopaque element at the distal end of the array and defining the distal extremity of the apparatus.

11. A device as defined in claim 8 further comprising:
the core tube defining a lumen to enable therapeutic substances to flow there through; and
at least one passageway extending between the interior of the core tube and the outer surface of the array whereby therapeutic substances may be delivered through the apparatus to a region immediately surrounding the array.

12. An apparatus as defined in claim 11 wherein the conductors are contained within the flow passage defined through the core tube.

13. An apparatus as defined in claim 11 further comprising an inner tubular member disposed within and extending along the core tube, the inner tube defining a flow lumen for therapeutic substances;
the conductors being disposed between the core tube and the inner tube, thereby isolating the conductors from the flow lumen of the inner tube.

14. A method for enhancing delivery of therapeutic substances to a region of tissue comprising:
providing a multiple-element array of coaxially aligned, acoustically isolated annular ultrasound elements, the array defining a fixed maximum outer diameter, each element having an outer radiating surface for emitting ultrasound in a radially outward pattern about the axis;
implanting the array in immediate proximity to target tissue;
activating the array to cause ultrasound energy to be emitted from the ultrasound elements in a phased manner adapted to cause constructive interference of pressure waves emitted from the transducer elements and in a manner that defines a focal ring and an annular range between the emission surface of the ultrasound element and the focal ring;
placing a therapeutic substance within the tissue encompassed within the annular range; and
operating the ultrasound element in a phased sequence to generate acoustic energy above a threshold level for inducing radially outward transport of molecules of therapeutic substance, the level of emitted energy being below a level to cause thermal damage to the tissue.

15. A method for enhancing the delivery of therapeutic substances to a region of tissue comprising:
generating a radially extending ultrasound field about a central axis, the field defining an annular range that includes a focal ring at a location spaced from the emission surface from which the ultrasound is emitted;
the field being defined by resultant pressure waves that increase in amplitude in a radially outward direction to the focal ring;
the amplitude of the pressure waves at the radially inner portion of the annular range being characterized by avoidance of thermal damage to tissue while generating sufficient acoustic pressure to induce radially outward propagation of a substance disposed within the annular range.

16. A method a defined in claim 15 further comprising sweeping the focal ring through the annular range.

17. A method for enhancing delivery of therapeutic substances to a region of tissue comprising:
providing a multiple-element array of coaxially aligned, acoustically isolated annular ultrasound elements, the array defining a fixed maximum outer diameter, each element having an outer radiating surface for emitting ultrasound in a radially outward pattern about the axis;
implanting the array in immediate proximity to target tissue;
activating the array to cause ultrasound energy to be emitted from the ultrasound elements in substantially identical phase to define a diffused annular ring about the array;
placing a therapeutic substance within the tissue encompassed within the annular range; and
operating the ultrasound elements to generate acoustic energy above a threshold level for inducing radially outward transport of molecules of therapeutic substance, the level of emitted energy being below a level to cause thermal damage to the tissue.
